(19) United States
(12) Patent Application Publication
(10) Pub. No.: US 2024/0285958 A1
(43) Pub. Date: Aug. 29, 2024

(54) IMPEDANCE TOMOGRAPHY SYSTEM FOR ALIGNMENT FEEDBACK OF A CHARGING DEVICE FOR AN IMPLANTABLE MEDICAL DEVICE

(71) Applicant: BIOTRONIK SE & Co. KG, Berlin (DE)

(72) Inventor: David McIntosh, Wilsonville, OR (US)

(21) Appl. No.: 18/572,014

(22) PCT Filed: Jul. 13, 2022
(86) PCT No.: PCT/EP2022/069540

§ 371 Date: Dec. 19, 2023

Related U.S. Application Data
(60) Provisional application No. 63/221,526, filed on Jul. 14, 2021.

(30) Foreign Application Priority Data
Aug. 10, 2021 (DE) 20 2021 104 268.0

(51) Int. Cl. A61N 1/378; A61N 1/372

(52) U.S. Cl. CPC A61N 1/3787; A61N 1/37258

(57) ABSTRACT

The present invention relates to a medical system (1) comprising an implantable medical device (2), comprising an energy storage device for supplying electrical energy to the medical device (2), and a secondary coil (20) for transferring electrical energy to the energy storage device, and a charging device (3) which is designed to charge the energy storage device, the charging device (3) having a primary coil (30) via which electrical energy can be transferred to the energy storage device via the secondary coil of the implantable medical device (2). According to the invention, it is provided that the charging device (3) has a plurality of metal electrodes (31) which are arranged in front of the primary coil (30) and are each assigned to a segment (32) of a housing surface (33) of the charging device (3), the charging device (3) being designed to use the metal electrodes (31) to calculate an impedance for each segment (32) and to use the impedances to check whether the primary coil (30) is aligned with the secondary coil (20) for optimum energy transfer.

The present invention relates to a medical system comprising an implantable medical device and an external charging device for charging an energy storage device of the medical device, for example in the form of an implantable pulse generator (IPG).

In a receiver for a resonant inductive system for wireless power transfer (WPT), a coil at the charging device excites a coil at the implantable medical device. The alignment of the coils on the charging device and the implantable medical device is critical for highly efficient energy transfer. Efficient energy transfer allows for less thermal stress on the patient and potentially faster charging times. Alignment feedback is also important for the patient to quickly align the charging device over the implantable or implanted medical device (in some cases in awkward locations such as the back).

One way to determine alignment is to identify the conductive metallic titanium housings typically used in implants (pacemakers, neurostimulators).

Impedance tomography is a technique that measures the impedance of a target object to infer the composition or presence of a material. Impedance measurements consist of an array of electrodes (at least two) to which an alternating current is output and the corresponding voltage is measured. According to Ohm's law, impedance is Z=V/I, where Vis the measured complex voltage and I is the injected complex current. Complex refers to the resistive and reactive components of the impedance. Based on the medium in which the current flows, the impedance changes. Impedance tomography is sensitive to electrical conductivity. In this regard, larger electrode arrays allow for finer resolution imaging.

In the prior art, arrays of magnetic field sensor coils are known to be located on the coil of the charging device. When the charging device is over a metal target, the magnetic field sensor coils change amplitude. Furthermore, a detection of the primary coil of the charging device (metal detector mode) is known. Furthermore, the presence of a backscatter (e.g. LSK) can also be detected.

Further, US 2018/0212451 A1 discloses charging an energy source located in an implantable medical device implanted in a patient, the charging device comprising first and second pairs of electric coils configured to generate first and second uniform magnetic fields in overlapping first and second cylindrical regions located between the respective pairs of electric coils.

Further, WO 2019/173866 A1 discloses a device for providing sensory stimulation to a subject, the device comprising a coil system having at least one coil and an electronic controller operating in accordance with software instructions. In use, the controller receives input signals, performs an analysis of the input signals, and uses the results of the analysis to cause a signal generator to generate stimulation signals, the stimulation signals being applied to the coil system to thereby generate a stimulating electromagnetic field in a target region of the subject.

Based on the prior art set forth above, the present invention is directed to implementing a means for providing rapid alignment feedback to a patient attempting to position an external charging device over their implant for optimal and efficient energy transfer.

This task is solved by a medical system having the features of claim 1. Advantageous embodiments of the invention are described below.

According to claim 1, a medical system is disclosed, comprising:
an implantable medical device comprising an energy storage device (e.g., in the form of a battery) for supplying electrical energy to the implantable medical device, and a secondary coil for transferring electrical energy to the energy storage device, and
an external or separate charging device designed for charging the energy storage device, the charging device comprising a primary coil via which electrical energy can be transferred to the energy storage device via the secondary coil of the implantable medical device.

According to the invention, it is provided that the charging device has a plurality of metal electrodes which are arranged in front of the primary coil and are each assigned to a segment of a housing surface of the charging device, the segments forming a two-dimensional field or array, and the charging device being designed to use the metal electrodes to calculate an impedance for each segment in order to check whether the primary coil is aligned with respect to the secondary coil for optimum energy transfer, i.e., is opposite the latter, in particular coaxially.

The system according to the invention thus enables the detection of the position of the implanted medical device and thus the possibility for the charging device to provide alignment feedback (direction and rough distance) to the patient. The invention thus promotes efficient energy transfer from the charging device to the implant. In other words, the charging device has an impedance tomography function to identify the position of the implant (e.g., IPG) and then provide alignment feedback to the user between the coils on both sides.

According to a preferred embodiment of the medical system according to the present invention, the charging device comprises at least two metal electrodes arranged on the housing surface and adapted to contact the skin of a patient.

Further, according to a preferred embodiment of the medical system according to the present invention, the housing surface is formed by a flat substrate connected to a rest of the charging device.

According to a preferred embodiment, the flat substrate is a patch or a plaster.

Further, according to a preferred embodiment of the medical system according to the present invention, the energy storing device is a battery.

Further, according to a preferred embodiment of the medical system according to the present invention, the charging device is adapted to induce an alternating current in the metal electrodes and to measure the corresponding alternating voltage induced by this current, wherein the charging device is further adapted to calculate the complex impedance (Z=V/I) from the alternating current I and the corresponding alternating voltage V for at least four segments 32, particularly for 16 segments of the housing surface.

Further, according to a preferred embodiment of the medical system according to the present invention, on the basis of images which indicate the impedances in the individual segments, a presence of conductors and non-conductors is distinguished by the medical system on the basis of the impedances to determine an orientation of the primary and secondary coils with respect to one another.

Further, according to a preferred embodiment of the medical system according to the present invention, the charging device is adapted to output user feedback for the purpose of aligning the primary coil over the secondary coil.

Further, according to a preferred embodiment of the medical system according to the present invention, the medical system is adapted to provide the user feedback via a hand-held remote control or via warning tones.

According to yet another aspect of the present invention, a medical system is disclosed, the medical system comprising:
an implantable medical device comprising an energy storage device for supplying electrical energy to the medical device and a secondary coil for transferring electrical energy to the energy storage device, and
a charging device which is designed to charge the energy storage device, the charging device comprising multiple primary coils in an array, wherein the medical system is adapted to use impedance tomography to determine which primary coil is optimal for activation to achieve high power transfer efficiency.

In the following, embodiments of the present invention and other features and advantages of the invention will be explained with reference to the Figures, wherein

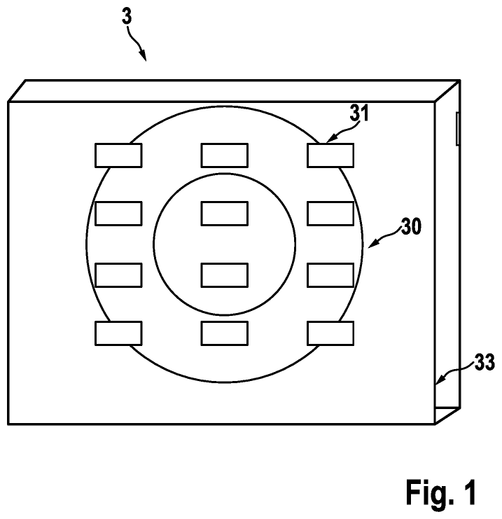

FIG. 1 shows a housing surface of a charging device of a system according to the invention, the housing surface comprising an array of segments, each segment having an associated metal electrode arranged in front of a primary coil of the charging device,

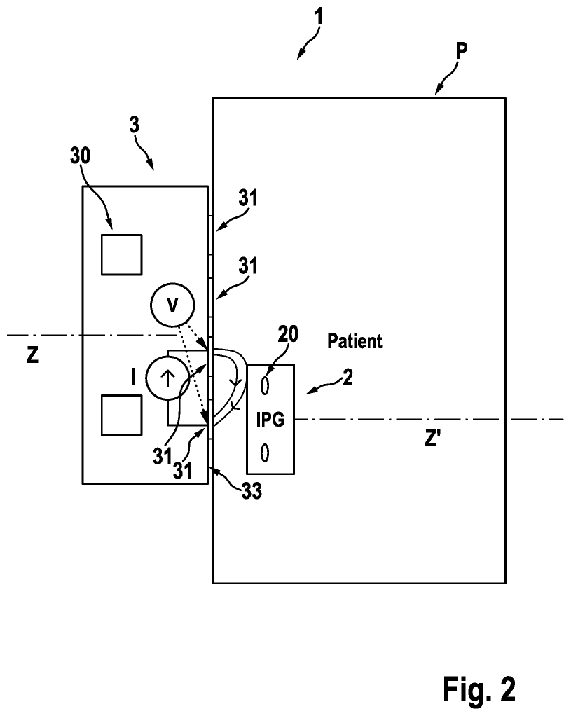

FIG. 2 shows a schematic sectional view of the charging device and implantable medical device (implanted in a patient), wherein the metal electrodes on the housing surface contact the skin surface of the patient,

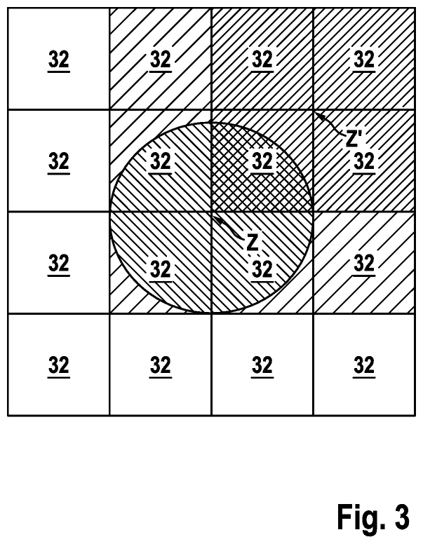

FIG. 3 shows the segments of the housing surface, although here there is still no optimal alignment of the charging device because the axes of the primary and secondary coils do not coincide, and

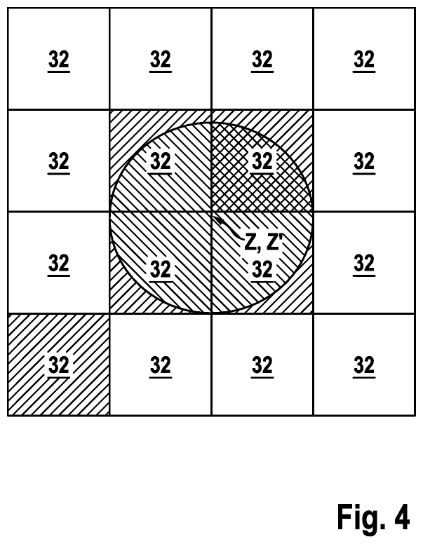

FIG. 4 shows an optimal alignment of the charging device with the coil axes of the primary and secondary coils coinciding.

FIG. 1 shows, in connection with FIG. 2, a medical system 1 according to the invention with a wireless power transfer (WPT) system comprising an external charging device 3 and an implantable medical device 2 (e.g. IPG), wherein the charging device 3 includes a primary coil 30, and wherein the implantable medical device 2 implanted in a patient P according to FIG. 2 includes a secondary coil 20.

The charging device 3 includes at least two metal electrodes 31 proximate to the body and adapted to contact the skin of the patient P. The electrodes 31 are arranged on a housing surface 33 of the charging device 3. The housing surface 33 may also be formed by a flat substrate (e.g. patch or plaster) suitably connected to the rest of the charging device 3.

Referring to FIG. 2, the charging device 3 is adapted to induce an alternating current I in the metal electrodes 31, the charging device 3 measuring the corresponding alternating voltage V induced by this current. From this, the charging device 3 calculates the complex impedance (Z=V/I) for at least four segments 32 or, in this case, for all segments 32 of the housing surface 33 shown in FIGS. 3 and 4. On the basis of these images, which indicate the impedances in the individual segments 32 and which are shown by way of example in FIGS. 3 and 4 for various orientations of the coils 20, 30 on both sides, the presence of conductors and non-conductors is distinguished on the basis of the impedances, so that it is possible to determine the orientation of the coils 20, 30 with respect to one another. Preferably, the charging device 3 is adapted to output user feedback for the purpose of aligning the charging device 3 or the primary coil 30 over the metallic implant 2 or its secondary coil 20. The user feedback can be provided, for example, via a hand-held remote control, via warning tones or the like.

FIG. 3 shows an example of an impedance image that results from calculating the impedances for the segments 32. In FIG. 3, the Z′ axis of the secondary coil 20 is too far up and to the right of the center or Z axis of the primary coil 30.

After alignment feedback by the charging device 3 to the patient P and patient movement of the charging device 3, the positioning changes to the optimal alignment shown in FIG. 4, where the coil axes Z, Z′ coincide.

Another embodiment of the invention features a charging device with multiple primary coils in an array. Impedance tomography is used to determine which coil is optimal for activation to achieve high power transfer efficiency. This is the opposite of the single primary coil moving to find the implant; here the correct coil is selected electronically.

The invention allows reliable identification of implant 2 compared to other methods that use multiple sense/search coils to look for disturbances in the excitation field. Impedance tomography may also prove useful for other reasons (e.g., to determine tissue response to the implant, particularly inflammation, scar tissue, etc.).

1. A medical system (1), comprising:
(a) an implantable medical device (2) comprising an energy storage device for supplying electrical energy to the medical device (2) and a secondary coil (20) for transferring electrical energy to the energy storage device;
(b) a charging device (3) designed to charge the energy storage device, the charging device having a primary coil (30) via which electrical energy can be transferred to the energy storage device via the secondary coil (20) of the implantable medical device (2),
wherein the charging device (3) comprises a plurality of metal electrodes (31) which are arranged in front of the primary coil (30) and are each assigned to a segment (32) of a housing surface (33) of the charging device (3), the charging device (3) being designed to use the metal electrodes (31) to calculate an impedance for each segment (32) and to use the impedances to check whether the primary coil (30) is aligned for optimum energy transfer to the secondary coil (20).

2. The medical system according to claim 1, wherein the charging device (3) comprises at least two metal electrodes (31) arranged on the housing surface (33) and adapted to contact the skin of a patient (P)

3. The medical system according to claim 1, wherein the housing surface (33) is formed by a flat substrate connected to a rest of the charging device (3).

4. The medical system according to claim 3, wherein the flat substrate is a patch or a plaster.

5. The medical system according to claim 1, wherein the energy storing device is a battery.

6. The medical system according to claim 1, wherein the charging device (3) is adapted to induce an alternating current (I) in the metal electrodes (31) and to measure the corresponding alternating voltage (V) induced by this current, wherein the charging device (3) is further adapted to calculate the complex impedance (Z=V/I) from the alternating current (I) and the corresponding alternating voltage (V) for at least four segments (32) or for 16 segments (32) of the housing surface (33).

7. The medical system according to claim 6, wherein on the basis of images which indicate the impedances in the individual segments (32) a presence of conductors and non-conductors is distinguished by the medical system on the basis of the impedances to determine an orientation of the primary and secondary coils (20, 30) with respect to one another.

8. The medical system according to claim 1, wherein the charging device (3) is adapted to output user feedback for the purpose of aligning the primary coil (30) over the secondary coil (20).

9. The medical system according to claim 8, wherein the medical system is adapted to provide the user feedback via a hand-held remote control or via warning tones.

10. A medical system, comprising:
(a) an implantable medical device comprising an energy storage device for supplying electrical energy to the medical device and a secondary coil for transferring electrical energy to the energy storage device; and
(b) a charging device designed to charge the energy storage device, the charging device comprising multiple primary coils in an array, wherein the medical system is adapted to use impedance tomography to determine which primary coil is optimal for activation to achieve high power transfer efficiency.
